(19) United States
(12) Patent Application Publication
(10) Pub. No.: US 2022/0273497 A1
(43) Pub. Date: Sep. 1, 2022

(54) TRANSFER METHOD FOR TREATING ABRASION WOUNDS WITH CYANOACRYLATE GLUE

(71) Applicant: Richard Sheldon Carlisle, Madrid, NY (US)

(72) Inventor: Richard Sheldon Carlisle, Madrid, NY (US)

(21) Appl. No.: 17/187,805

(22) Filed: Feb. 28, 2021

(51) Int. Cl. A61F 13/02; A61L 24/06

(52) U.S. Cl. CPC A61F 13/024; A61F 13/0266; A61L 24/06; A61F 13/0253; A61L 2300/606; A61L 2400/04; A61F 2013/00157

(57) ABSTRACT

A method for treating burns and abrasion wounds by applying a cyanoacrylate glue, using a method whereby a quantity of glue is initially applied to a flexible film of releasable material that does not adhere to the glue after it has set, and then transferring said glue to a wound site with light pressure applied through said film sufficient to control bleeding. Said application of glue under pressure will result in a thin layer of glue over the wound site that will react with the moisture present on the wound surface, thus polymerizing quickly and forming a protective coating on the wound that is flexible and sufficient to resist pathogenic penetration while simultaneously allowing the slow passage of moisture at a rate that will maintain an ideal relatively dry condition for the wound, to prevent infections that tend to develop with excessive moisture.

A method of treating abrasion wounds is provided that offers a very quick and effective protection for abrasion wounds in addition to burns that present similarly to abrasions, i.e., where epidermal layers are missing.

The method of treating said wounds consists of placement of an amount of cyanoacrylate glue on a flexible film that does not adhere to glue and transferring said glue to a wound site with light pressure sufficient to control bleeding. The pressure application can be sustained for about five minutes to allow the glue to polymerize under the film after contact with the slight amount of moisture on the wound surface. Removing said pressure and said film can be done after polymerization.

Said flexible release film can be temporarily wrapped around a fingertip as a way of presenting the film for coating with said glue for treatment of small wounds up to an inch in diameter, but for wounds that are substantially larger, then a form in the shape of a chicken egg, similarly varying in spherical contour can be used to temporarily wrap the film around while glue is being applied to the film. Also, in applying the glue, it should be deposited first on a surface such as the bottom of a small container and then the flexible film wrapped around the form can be dipped into the glue, to neatly apply an ideal amount to the film. When a form used is egg shaped, the variable spherical contour allows selecting a region of the form that best matches the size and shape of the wound. Then, placing the egg-shaped object over the abrasion wherein the glue side of the film contacts the abrasion, and applying pressure to the film with the egg-shaped object sufficient to control bleeding, the final step is removing said egg-shaped object and flexible release film. After treating an abrasion with the method described, there will be a lack of inflammation during healing, which will further result in an ideal cosmetic repair.

1. A method for treating abrasion and burn wounds with a cyanoacrylate glue comprising,
(a) applying said cyanoacrylate glue to a flexible layer of release film that does not adhere to said glue, and
(b) transferring said glue to a wound surface with sufficient pressure applied through said film to control bleeding while maintaining a moist surface on the wound area,
(c) allowing a time period of up to five minutes for said glue to react with said moisture and polymerize, and
(d) allowing a further time period for the wound to heal with minimal inflammation and produce an ideal cosmetic result.

2. A method for treating an abrasion wound comprising the steps,
(a) applying a cyanoacrylate glue to a flexible release film,
(b) transferring said glue to the wound using pressure sufficient to control bleeding, wherein the pressure is applied through said film while maintaining a moist wound surface,
(c) waiting for a time period of five minutes for said glue to react with said moisture and polymerize, and
(d) removing said pressure and said film.

3. A method for applying a cyanoacrylate glue to an abrasion comprising,
(a) wrapping a flexible release film around an egg-shaped object, said egg-shaped object having a variable spherical contour,
(b) applying a cyanoacrylate glue to the flexible release film,
(c) placing the egg-shaped object over an abrasion wherein a side of the film having glue contacts the abrasion,
(d) applying pressure to the film with the egg-shaped object, wherein an amount of pressure applied is sufficient to control bleeding,
(e) maintaining pressure until said cyanoacrylate glue has polymerized, and
(f) removing said egg-shaped object and flexible release film.

4. (canceled)
